(19) United States
(12) Patent Application Publication
(10) Pub. No.: US 2025/0180485 A1
(43) Pub. Date: Jun. 5, 2025

(54) BATTERY MONITORING APPARATUS

(71) Applicants: Hyundai Motor Company, Seoul (KR); Kia Corporation, Seoul (KR); CHUNG ANG University Industry Academic Cooperation Foundation, Seoul (KR)

(72) Inventors: Seung Min OH, Incheon (KR); Sung Ho Ban, Hwaseong-Si (KR); Sang Hun Lee, Paju-Si (KR); Chang Hoon Song, Seoul (KR); Tae Ho Park, Hwaseong-Si (KR); Eung Ju Lee, Daejeon (KR); Ki Kang Lee, Suwon-Si (KR); Yoon Sung Lee, Suwon-Si (KR); Seung Tae Kim, Yongin-Si (KR); Hyung Bin Son, Seoul (KR); Hyunchul Kang, Seoul (KR); kibeom Hwang, Seoul (KR); Seok-In Kim, Seoul (KR); Songhun Yoon, Seoul (KR)

(73) Assignees: Hyundai Motor Company, Seoul (KR); Kia Corporation, Seoul (KR); CHUNG ANG University Industry Academic Cooperation Foundation, Seoul (KR)

(21) Appl. No.: 18/799,331

(22) Filed: Aug. 9, 2024

(30) Foreign Application Priority Data
Dec. 5, 2023 (KR) 10-2023-0174744

(51) Int. Cl. G01N 21/65; G01N 33/00; H01M 10/42; H01M 10/48

(52) U.S. Cl. CPC G01N 21/65; G01N 33/0027; H01M 10/4285; H01M 10/488

(57) ABSTRACT

A battery monitoring apparatus may be used for gas analysis of medium to large-sized actual cells by allowing the gases generated during charging and discharging of lithium secondary batteries to be analyzed in-situ through Raman analysis equipment without external exposure of the gas. Meanwhile, the battery monitoring apparatus can identify relationship between a change in cell volume and a battery performance due to gas generation by measuring the change in cell volume and gas pressure according to the gas generated during the cycle, and can also measure the safety and life predictability of the actual cell by identifying the correlation with residual useful life through analysis of electrochemical results and gases generated according to cycle performance.

### CROSS REFERENCE TO RELATED APPLICATION

The present application claims priority to Korean Patent Application No. 10-2023-0174744, filed Dec. 5, 2023, the entire contents of which is incorporated herein for all purposes by this reference.

### BACKGROUND OF THE PRESENT DISCLOSURE

### Field of the Present Disclosure

The present disclosure relates to a battery monitoring apparatus which may be used for gas analysis of medium to large-sized actual cells by allowing the gases generated during charging and discharging of lithium secondary batteries to be analyzed in-situ through Raman analysis equipment without external exposure of the gas, can identify relationship between a change in cell volume and a battery performance due to gas generation by measuring the change in cell volume and gas pressure according to the gas generated during the cycle, and can also measure the safety and life predictability of the actual cell by identifying the correlation with residual useful life through analysis of electrochemical results and gases generated according to cycle performance.

### Description of Related Art

Lithium secondary batteries are widely used in portable energy storage devices and electric vehicles due to their high energy density, low cost, long cycle life, and safety. The development of lithium-ion batteries with high energy density and long lifespan is attracting attention. In modern society, these devices are used as energy sources not only for portable devices but also for hybrid vehicles, electric vehicles, and ESS, and their lifespan is emerging as an important issue. Among them, lithium secondary batteries with high energy density are attracting attention as backup power devices for electric vehicles and generators. However, because the lifespan of these lithium secondary batteries varies depending on the operating environment, they cannot be used below 80% of their initial capacity, which is their guaranteed lifespan. Therefore, there is a demand for a diversified analysis of the causes of cell degradation due to charging/discharging.

However, conventional gas generation researches have mainly been conducted in the direction of studying gas generation and conversion mechanisms of gases in abnormal situations such as high temperature, after cycling, and overcharge, and the gas generation phenomenon in the charging state has not been studied through in-situ analysis.

The information included in this Background of the present disclosure is only for enhancement of understanding of the general background of the present disclosure and may not be taken as an acknowledgement or any form of suggestion that this information forms the prior art already known to a person skilled in the art.

### BRIEF SUMMARY

Various aspects of the present disclosure are directed to enabling in-situ analysis of gases generated during charging and discharging of lithium secondary batteries using Raman analysis equipment without external exposure.

Furthermore, the present disclosure is to be used for gas analysis of medium to large-sized actual cells in the future.

Meanwhile, the present disclosure is to identify relationship between a change in cell volume and a battery performance due to gas generation by measuring the change in cell volume and gas pressure according to the gas generated during the cycle.

Furthermore, the present disclosure is to measure the safety and life predictability of the actual cell by identifying the correlation with residual useful life through analysis of electrochemical results and gases generated according to cycle performance.

The technical objects to be achieved as an exemplary embodiment of the present disclosure are not limited to the technical objects mentioned above, and other technical objects not mentioned may be clearly understood by those skilled in the art from the following descriptions.

In various aspects of the present disclosure, a battery monitoring apparatus of the present disclosure may include a packaging material in which a battery cell is built thereinside, an observation space where gas is introduced is formed on one side of a battery, and an observation hole is formed at a point where the observation space is formed; and a sealing portion which is attached to the observation hole of the packaging material to seal the observation hole, and is made of a material that transmits a laser beam so that the gas in the observation space is able to be analyzed through the laser beam.

For example, the packaging material is a film that forms a pouch of the battery cell and extends to one side of the battery monitoring apparatus to form the observation space.

For example, the observation holes are formed on first and second sides of the packaging material forming the observation space, respectively, and the observation holes on the first and second sides are formed at positions facing each other.

For example, the sealing portion is made of any one of glass, plastic, and a mixture thereof.

For example, the sealing portion and the packaging material are coupled by thermal sealing.

For example, the apparatus further includes a support which is provided inside the observation space and supports the observation space.

For example, the support is made of any one of chalcedony, agate, onyx, jasper, aventurine, tiger's eye, amethyst, citrine, rose quartz, milk quartz, soft quartz, black quartz, cornelian, flint, and a mixture thereof.

For example, the support is in a shape of a pillar extending in a vertical direction of the apparatus.

For example, a transmission hole through which the laser beam passes is formed inside the support, and the transmission hole faces the observation hole.

For example, the transmission hole extends in a vertical direction of the apparatus and is formed in a position corresponding to the observation hole in the packaging material.

For example, a distribution hole is formed inside the support, and the distribution hole extends from an external surface of the support and is connected to the transmission hole, so that the gas generated from the battery cell is introduced into the transmission hole through the distribution hole.

For example, the apparatus further includes a washer which is coupled to an outside of the packaging material and covers the observation hole.

For example, a washer hole is formed in an upper portion of the washer at a position corresponding to the observation hole to guide measurement equipment therethrough.

For example, the washer is made of any of PP, PE, PS, PET, PA, PES, PVC, PU, PC, PVDC and a mixture thereof.

For example, a perforated hole is formed in the packaging material at a point in contact with the washer, and the perforated hole is filled with an adhesive to combine the washer, the packaging material, and the sealing portion.

For example, a plurality of perforated holes is formed spaced apart in a direction surrounding the observation hole.

For example, the adhesive hardens in response to ultraviolet ray and does not leak out of the perforated hole, so that the adhesive does not react with the gas or electrolyte generated from the battery cell.

For example, the apparatus further includes a gas permeable membrane which is located between the battery cell and the observation space, and allows the gas and other substance generated from the battery cell to penetrate into the observation space.

The gases generated during charging and discharging of lithium secondary batteries may be analyzed in-situ using Raman analysis equipment without external exposure. Furthermore, it may be used for gas analysis of medium to large-sized real cells in the future.

Furthermore, it can identify the relationship between a change in cell volume and a battery performance due to gas generation by measuring the change in cell volume and gas pressure according to the gas generated during the cycle.

Meanwhile, through analysis of electrochemical results and gases generated according to cycle performance, the correlation with residual useful life may be identified, and the safety and life predictability of the actual cell may be measured.

The effects of the present disclosure are not limited to the above-described effects and other effects which are not described herein will become apparent to those skilled in the art from the following description.

The methods and apparatuses of the present disclosure have other features and advantages which will be apparent from or are set forth in more detail in the accompanying drawings, which are incorporated herein, and the following Detailed Description, which together serve to explain certain principles of the present disclosure.

### BRIEF DESCRIPTION OF THE DRAWINGS

FIG. 1 is a diagram showing a battery monitoring apparatus of the present disclosure.

FIG. 2 is a diagram showing a portion of a battery monitoring apparatus of the present disclosure.

FIG. 3 is a diagram showing an example of a battery monitoring apparatus of the present disclosure.

It may be understood that the appended drawings are not necessarily to scale, presenting a somewhat simplified representation of various features illustrative of the basic principles of the present disclosure. The specific design features of the present disclosure as included herein, including, for example, specific dimensions, orientations, locations, and shapes will be determined in part by the particularly intended application and use environment.

In the figures, reference numbers refer to the same or equivalent parts of the present disclosure throughout the several figures of the drawing.

### DETAILED DESCRIPTION

Reference will now be made in detail to various embodiments of the present disclosure(s), examples of which are illustrated in the accompanying drawings and described below. While the present disclosure(s) will be described in conjunction with exemplary embodiments of the present disclosure, it will be understood that the present description is not intended to limit the present disclosure(s) to those exemplary embodiments of the present disclosure. On the other hand, the present disclosure(s) is/are intended to cover not only the exemplary embodiments of the present disclosure, but also various alternatives, modifications, equivalents and other embodiments, which may be included within the spirit and scope of the present disclosure as defined by the appended claims.

Hereinafter, various exemplary embodiments of the present disclosure will be described in detail with reference to the appended drawings. The same or similar components are provided the same reference numbers and redundant description thereof is omitted.

In describing the exemplary embodiment included in the specification, when a detailed description of known techniques associated with the present disclosure would unnecessarily obscure the gist of the present disclosure, detailed description thereof will be omitted. Furthermore, the appended drawings are provided for easy understanding of embodiments of the present disclosure and do not limit technical spirits of the present disclosure, and the exemplary embodiments should be construed as including all modifications, equivalents, and alternatives falling within the spirit and scope of the embodiments.

The singular forms are intended to include the plural forms as well, unless the context clearly indicates otherwise.

In the specification, it will be further understood that the terms “comprise” and “include” specify the presence of stated features, integers, steps, operations, elements, components, or combinations thereof, but do not preclude the presence or addition of one or more other features, integers, steps, operations, elements, components, or combinations.

The suffixes “module” and “unit” of elements herein are used for convenience of description and thus may be used interchangeably and do not have any distinguishable meanings or functions.

When an element is “coupled” or “connected” to another element, it should be understood that a third element may be present between the two elements although the element may be directly coupled or connected to the other element. When an element is “directly coupled” or “directly connected” to another element, it should be understood that no element is present between the two elements.

Raman spectroscopy utilizes the Raman effect. When a laser is fired at a specific molecule, Raman spectroscopy is an experimental method that measures the surface state of the molecule through the phenomenon of absorbing energy equal to the difference in the electron energy levels of the molecule, and measures the degree of fragmentation of the sample through the measurements. Specifically, it can be measured using a Raman spectrometer (ReactRaman 802L, METTLER TOLEDO. Inc).

According to an exemplary embodiment of the present disclosure, in-situ analysis of gases generated during charging and discharging of lithium secondary batteries may be performed using Raman analysis equipment without external exposure. Furthermore, the present disclosure may be used for gas analysis of medium to large-sized actual cells in the future. Furthermore, it is possible to identify the relationship between a change in cell volume and a battery performance due to gas generation by measuring the change in cell volume and gas pressure according to the gas generated during the cycle. Meanwhile, through the analysis of electrochemical results and gases generated according to cycle performance, the correlation with residual useful life may be identified, and the safety and life predictability of the actual cell may be measured. The present disclosure relates to a battery monitoring apparatus having these effects.

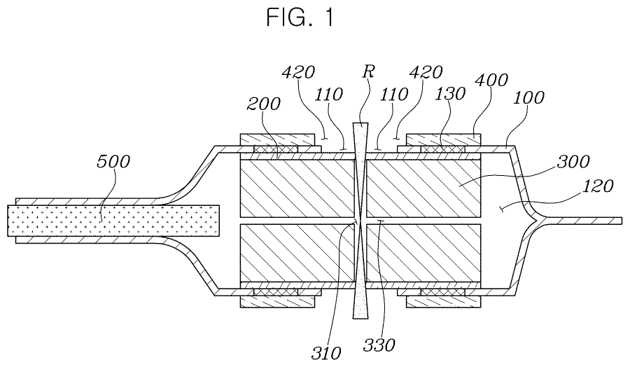

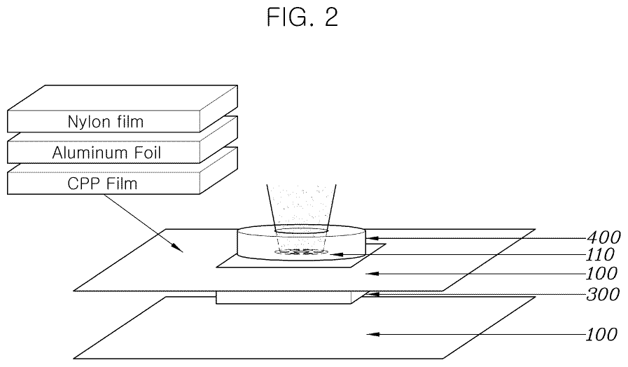

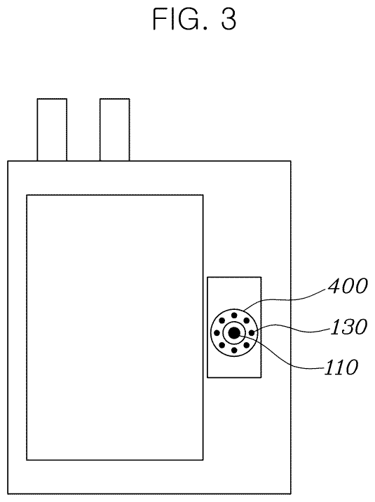

FIG. 1 is a diagram showing a battery monitoring apparatus of the present disclosure, FIG. 2 is a diagram showing a portion of a battery monitoring apparatus of the present disclosure, FIG. 3 is a diagram showing an example of a battery monitoring apparatus of the present disclosure.

FIG. 1 is a diagram showing a battery monitoring apparatus of the present disclosure, and FIG. 2 is a diagram showing a portion of a battery monitoring apparatus of the present disclosure. With reference to FIG. 1 and FIG. 2, a battery monitoring apparatus of the present disclosure will be described.

The packaging material 100 of the battery motoring apparatus of the present disclosure is an external material for a battery and may have battery cells built therein, and may be extended to one side to form an observation space 120.

In the instant case, the packaging material 100 may be an external material for a cylindrical battery, a square battery, or a pouch-type battery. The packaging material 100 is an external material of a pouch-type battery, as shown in FIG. 2, and may include a form in which nylon film, aluminum foil, and CPP film are stacked.

Through the present structure, an observation space 120 may be formed simply by extending the packaging material 100, which is the external material of the battery, without a separate complicated structure. Furthermore, because the battery cell and observation space 120 are integrated into the packaging material 100, a vulnerable area where gas generated from the battery cell leaks to the outside may be effectively reduced.

An observation hole 110 is formed in the packaging material 100.

In the instant case, the shape of the observation hole 110 may be circular, oval, or polygonal including a square. However, this is an example and is not limited to a specific shape, as long as it includes an external Raman measurement device and includes a shape suitable for transmitting the laser of an optical measurement device.

The observation holes 110 are formed on both opposing sides of the packaging material 100 forming the observation space 120, and the observation holes 110 on both sides are formed at positions facing each other.

Through the present structure, the light beam (R) including the laser of an optical device irradiated from the outside thereof may be effectively irradiated into the observation space 120, which allows the gas generated from the battery cell to be effectively monitored.

A sealing portion 200 is coupled to the observation hole 110 of the packaging material 100 to seal the observation hole 110, and is made of a material that transmits laser.

Herein, the sealing portion 200 may include any one of glass, plastic, and mixtures thereof.

Additionally, the sealing portion 200 and packaging material 100 may be joined by thermal sealing. In the instant case, thermal sealing may be performed in vacuum conditions for 1.5 to 3 seconds to remove gas and other impurities. Then, heating may be performed for 1.5 to 3 seconds at a sealing temperature of 150 to 200° C., and then cooling may be performed for more than 5 seconds at a temperature of 20 to 30° C.

Through the present structure, the sealing portion 200 effectively seals the observation hole 110 of the packaging material 100 and simultaneously removes gases inside the observation space 120. Accordingly, the sealing portion 200 can smoothly transmit a light beam R, including the laser irradiated from the outside of the observation space 120 while minimizing noise factors during battery monitoring.

A support 300 is provided inside the observation space 120 to support the observation space 120, and may be located between two opposing sides of the packaging material 100.

The support 300 may be made of any one of chalcedony, agate, onyx, jasper, aventurine, tiger's eye, amethyst, citrine, rose quartz, milk quartz, soft quartz, black quartz, cornelian, flint, and mixtures thereof, and may be made of metal material. However, this is an example and any material with hardness that can support the observation space 120 may be used as the support 300.

The support 300 may be in the shape of a pillar extending in the vertical direction on both opposing sides of the packaging material 100. Additionally, the cross-section of the support 300 in a direction perpendicular to the vertical direction may be any one of a polygon including a triangle, a square, and a pentagon, a circle, and an ellipse.

Through the present structure, the observation space 120 in the packaging material 100 may be stably secured and supported, and the observation space 120 may be effectively protected from external shock.

Meanwhile, a transmission hole 310 is formed inside the support 300 through which a light beam R including a laser passes, and the transmission hole 310 may be formed to face the observation hole 110 of the packaging material 100.

For example, as shown in FIG. 1, the transmission hole 310 may extend in the vertical direction and may be formed at a position corresponding to the observation hole 110 of the packaging material 100.

Additionally, a distribution hole 330 may be formed inside the support 300. The distribution hole 330 may extend from the external surface of the support 300 and be connected to the transmission hole 310.

For example, as shown in FIG. 1, the distribution hole 330 may extend laterally to both end portions perpendicular to the vertical direction within the support body 300 and be connected to the transmission hole 310.

Through the present structure, a space is secured inside the support 300 through which the light beam R including the laser may be transmitted, and at the same time, the light beam R may be effectively concentrated into the transmission hole 310, resulting in effective monitoring of the gases generated from battery cells through a small amount of light R. Furthermore, the gas generated from the battery cell may be smoothly supplied to the transmission hole 310 located inside the support 300 through the distribution hole 330.

A washer 400 is coupled to the outside of the packaging material 100 and may be coupled to cover the observation hole 110.

Additionally, the washer 400 may be column-shaped, and its cross-section may be any one of a polygon including a triangle, square, and pentagon, a circle, and ellipse. This is an example and the cross-section may have any shape as long as it can cover the observation hole 110.

Through the present structure, the observation hole 110 of the packaging material 100 and sealing portion 200 may be effectively protected from external shock.

Furthermore, a washer hole 420 may be formed in the upper portion of the washer 400 at a position corresponding to the observation hole 110, and the size of the hole is so that the measurement portion of the measurement equipment including the Raman analyzer may be effectively guided.

Through the present structure, the measurement equipment including a Raman analyzer may be effectively guided and the gases generated from battery cells located in the observation space 120 may be analyzed.

The washer 400 may be made of a material including any one of PP, PE, PS, PET, PA, PES, PVC, PU, PC, PVDC, and mixtures thereof.

Meanwhile, as shown in FIG. 1, a perforated hole 130 may be formed at a point in contact with the washer 400 of the packaging material 100, and the perforated hole 130 is filled with an adhesive to combine the washer 400 and the packaging material 100 and sealing portion 200 through the adhesive.

Through the present structure, the washer 400 and packaging material 100 may be effectively coupled together through the adhesive. In the case of the packaging material 100 and sealing portion 200, they are coupled more strongly than through thermal sealing alone, effectively sealing a space between the outside and the observation space 120. Additionally, by preventing the adhesive from leaking out of the perforated hole 130 and reacting with gas or electrolyte solution generated from the battery, it can help increase chemical resistance.

Herein, a plurality of perforated holes 130 may be formed to be spaced apart in a direction surrounding the observation hole 110. For example, the plurality of perforated holes may be formed continuously and spaced apart to surround the observation hole 110 in the shape of a circle or a polygon including a square.

Through the present structure, the outside and observation space 120 may be effectively sealed.

In the instant case, the adhesive may be a UV adhesive that hardens in response to ultraviolet rays, or may be an ultraviolet adhesive that hardens in reaction to ultraviolet rays with a wavelength of 300 to 500 nm.

Through these characteristics, the adhesive hardens and is prevented from leaking out of the perforated hole 130, so that the adhesive is prevented from reacting with the gas or electrolyte solution generated from the battery, which can help increase chemical resistance.

A gas permeable membrane 500 is located between the battery cell and the observation space 120, and may allow the gas and other substances generated from the battery cell to penetrate into the observation space 120.

Through the present structure, it is possible to effectively monitor the battery by separating the battery cell and the observation space 120 while allowing the gases generated from the battery cell to penetrate into the observation space 120.

In an exemplary embodiment of the present disclosure, the perforated hole 130 is in plural and one of the plurality of perforated holes 130 is positioned on the first side and another of the plurality of perforated holes is positioned on the second side of the packaging material 100.

In an exemplary embodiment of the present disclosure, the observation hole 110 is in plural and the plurality of observation holes 110 are formed on first and second sides of the packaging material 100 forming the observation space 120, respectively, wherein the perforated hole 130 is in plural and one of the plurality of perforated holes 130 is positioned on the first side and another of the plurality of perforated holes 130 is positioned on the second side, and wherein the transmission hole 310 is formed inside the support 300, and the transmission hole 310 extends between the first and second sides of the packaging material 100.

Hereinafter, Examples and Comparative Examples are described to explain an effect of the battery monitoring mechanism of the present disclosure.

### Example 1

One extending side of a pouch battery in its pre-formation state is cut and opened, and an observation hole is formed in the packaging material.

Eight circular holes are formed at regular intervals around the observation hole.

After attaching a sealing portion to the inside of the packaging material, a quartz support is provided.

After injecting UV Bond into a perforated hole, a plastic washer is attached to the outside of the packaging material and adhered closely. Accordingly, the UV bond injected into the perforated hole is hardened by exposing the washer edge portion to ultraviolet rays with a wavelength of 380 nm through a UV lantern.

Using a vacuum sealer, vacuum is performed with a vacuum time of 0.3 seconds, sealing (heating wire) is performed at 180° C. for 2 seconds, and wind is injected through a cooling fan. Then, under the condition of more than 5 seconds at room temperature (25° C.), the work of removing the air that has already flowed in to the outside and sealing are ensured at the same time.

### Comparative Example 1

In Example 1, sealing was performed only by thermal sealing using a vacuum sealer without adhesive. A plastic washer is also not provided.

### Comparative Example 2

In Example 1, the packaging material and sealing portion were attached using an adhesive without forming a perforated hole, and then sealing was performed through thermal sealing using a vacuum sealer. The plastic washer is not provided.

Raman analysis is directly performed in real-time in-situ through a 10× lens barrel using a green laser with a wavelength of 532 nm and an intensity of 100 mW.

Then, after completing the production of the pouch battery, a first formation was performed by applying a voltage of 4.3V to the cells stored at room temperature for 24 hours, and then charging/discharging at 2.3V to 4.3V was performed 20 times at 4-hour intervals to monitor the gas generated in the battery cell.

Afterwards, experiments were conducted on Experimental Examples and Comparative Examples of the battery monitoring apparatus for batteries in a charged state. The results of these experiments are shown in Table 1 below.

TABLE 1

| Reinforcement | | Comparative | Comparative |
|---|---|---|---|
| method | Example | Example 1 | Example 2 |
| Airtightness | Excellent | Excellent | Excellent |
| Chemical resistance | Excellent | Excellent | Poor |
| Adhesion | Excellent | Poor | Excellent |
| Reproducibility | Excellent | Poor | Poor |

In case of airtightness, both Example and Comparative Examples 1 and 2 showed excellent results. Through this, it may be seen that a sufficient degree of airtightness is secured only by thermal sealing. In case of chemical resistance, Example and Comparative Example 1 show excellent result, but Comparative Example 2 shows poor result. Through this, it may be seen that when the adhesive is applied between the packaging material and the sealing portion without filling the adhesive into the perforated hole, the adhesive may react with the gas or electrolyte solution generated from the battery cell.

In the case of adhesion, it may be seen that Example and Comparative Example 2 show excellent result, and Comparative Example 1 shows poor result. Through this, it may be seen that when sealing is performed only by thermal sealing without adhesive, peeling may occur even with a slight external force.

In case of reproducibility, it may be seen that the Example shows excellent result, and Comparative Examples 1 and 2 show poor result. Through this, it may be seen that both the adhesive filled in the perforated hole and the plastic washer may be provided to effectively secure reproducibility even in repeated measurements.

Accordingly, the battery monitoring apparatus of the present disclosure can perform in-situ analysis of the gases generated during charging and discharging of lithium secondary batteries through Raman analysis equipment without external exposure, and may be used for gas analysis of medium to large-sized actual cells in the future. Accordingly, the present disclosure can provide an excellent battery monitoring apparatus capable of measuring the safety and life predictability of actual cells.

For convenience in explanation and accurate definition in the appended claims, the terms “upper”, “lower”, “inner”, “outer”, “up”, “down”, “upwards”, “downwards”, “front”, “rear”, “back”, “inside”, “outside”, “inwardly”, “outwardly”, “interior”, “exterior”, “internal”, “external”, “forwards”, and “backwards” are used to describe features of the exemplary embodiments with reference to the positions of such features as displayed in the figures. It will be further understood that the term “connect” or its derivatives refer both to direct and indirect connection.

The term “and/or” may include a combination of a plurality of related listed items or any of a plurality of related listed items. For example, “A and/or B” includes all three cases such as “A”, “B”, and “A and B”.

In the present specification, unless stated otherwise, a singular expression includes a plural expression unless the context clearly indicates otherwise.

In exemplary embodiments of the present disclosure, “at least one of A and B” may refer to “at least one of A or B” or “at least one of combinations of at least one of A and B”. Furthermore, “one or more of A and B” may refer to “one or more of A or B” or “one or more of combinations of one or more of A and B”.

In the exemplary embodiment of the present disclosure, it should be understood that a term such as “include” or “have” is directed to designate that the features, numbers, steps, operations, elements, parts, or combinations thereof described in the specification are present, and does not preclude the possibility of addition or presence of one or more other features, numbers, steps, operations, elements, parts, or combinations thereof.

According to an exemplary embodiment of the present disclosure, components may be combined with each other to be implemented as one, or some components may be omitted.

The foregoing descriptions of specific exemplary embodiments of the present disclosure have been presented for purposes of illustration and description. They are not intended to be exhaustive or to limit the present disclosure to the precise forms disclosed, and obviously many modifications and variations are possible in light of the above teachings. The exemplary embodiments were chosen and described in order to explain certain principles of the invention and their practical application, to enable others skilled in the art to make and utilize various exemplary embodiments of the present disclosure, as well as various alternatives and modifications thereof. It is intended that the scope of the present disclosure be defined by the Claims appended hereto and their equivalents.

What is claimed is:

1. A battery monitoring apparatus, comprising:
a packaging material in which a battery cell is built thereinside, an observation space where gas is introduced is formed on one side of a battery, and an observation hole is formed at a point where the observation space is formed; and
a sealing portion which is attached to the observation hole of the packaging material to seal the observation hole, and is made of a material that transmits a laser beam so that the gas in the observation space is able to be analyzed through the laser beam.

2. The apparatus of claim 1, wherein the packaging material is a film that forms a pouch of the battery cell and extends to one side of the battery monitoring apparatus to form the observation space.

3. The apparatus of claim 1, wherein the observation hole is in plural and the plurality of observation holes are formed on first and second sides of the packaging material forming the observation space, respectively, and the observation holes on the first and second sides are formed at positions facing each other.

4. The apparatus of claim 3, wherein the perforated hole is in plural and one of the plurality of perforated holes is positioned on the first side and another of the plurality of perforated holes is positioned on the second side.

5. The apparatus of claim 1, wherein the sealing portion is made of one of glass, plastic, and a mixture thereof.

6. The apparatus of claim 1, wherein the sealing portion and the packaging material are coupled by thermal sealing.

7. The apparatus of claim 1, further including a support which is provided inside the observation space and supports the observation space.

8. The apparatus of claim 7, wherein the support is made of one of chalcedony, agate, onyx, jasper, aventurine, tiger's eye, amethyst, citrine, rose quartz, milk quartz, soft quartz, black quartz, cornelian, flint, and a mixture thereof.

9. The apparatus of claim 7, wherein the support is in a shape of a pillar extending in a vertical direction of the apparatus.

10. The apparatus of claim 7, wherein a transmission hole through which the laser beam passes is formed inside the support, and the transmission hole faces the observation hole.

11. The apparatus of claim 10,
wherein the observation hole is in plural and the plurality of observation holes are formed on first and second sides of the packaging material forming the observation space, respectively,
wherein the perforated hole is in plural and one of the plurality of perforated holes is positioned on the first side and another of the plurality of perforated holes is positioned on the second side, and
wherein the transmission hole is formed inside the support, and the transmission hole extends between the first and second sides of the packaging material.

12. The apparatus of claim 7, wherein the transmission hole extends in a vertical direction of the apparatus and is formed in a position corresponding to the observation hole in the packaging material.

13. The apparatus of claim 7, wherein a distribution hole is formed inside the support, and the distribution hole extends from an external surface of the support and is connected to the transmission hole, so that the gas generated from the battery cell is introduced into the transmission hole through the distribution hole.

14. The apparatus of claim 1, further including a washer which is coupled to an outside of the packaging material and covers the observation hole.

15. The apparatus of claim 14, wherein a washer hole is formed in an upper portion of the washer at a position corresponding to the observation hole to guide measurement equipment therethrough.

16. The apparatus of claim 14, wherein the washer is made of any of PP, PE, PS, PET, PA, PES, PVC, PU, PC, PVDC and a mixture thereof.

17. The apparatus of claim 14, wherein a perforated hole is formed in the packaging material at a point in contact with the washer, and the perforated hole is filled with an adhesive to combine the washer, the packaging material, and the sealing portion.

18. The apparatus of claim 17, wherein the perforated hole is in plural and the plurality of perforated holes is formed spaced apart in a direction surrounding the observation hole.

19. The apparatus of claim 17, wherein the adhesive hardens in response to ultraviolet ray and does not leak out of the perforated hole, so that the adhesive does not react with the gas or electrolyte generated from the battery cell.

20. The apparatus of claim 1, further including a gas permeable membrane which is located between the battery cell and the observation space, and allows the gas and other substance generated from the battery cell to penetrate into the observation space.
